(19) United States
(12) Patent Application Publication
(10) Pub. No.: US 2016/0361115 A1
(43) Pub. Date: Dec. 15, 2016

(54) SALINE BETWEEN TIP AND TISSUE

(71) Applicant: Boston Scientific Scimed Inc., Maple Grove, MN (US)

(72) Inventors: Robert F. Bencini, Sunnyvale, CA (US); Ajitkumar B. Nair, Pleasanton, CA (US); Dushyant J. Shah, San Ramon, CA (US); Simon N.H. Nguyen, San Jose, CA (US); Jeffrey A. Sarge, Fremont, CA (US); Kevin L. Bencini, Sunnyvale, CA (US)

(21) Appl. No.: 15/177,682

(22) Filed: Jun. 9, 2016

Related U.S. Application Data
(60) Provisional application No. 62/173,763, filed on Jun. 10, 2015.

(51) Int. Cl. A61B 18/14

(52) U.S. Cl. CPC A61B 18/1492; A61B 2018/00065

(57) ABSTRACT

An ablation catheter assembly including a source of ablation energy coupled to a lead wire disposed within an elongated catheter shaft having a proximal end and a distal end, the lead wire having an electrode disposed at the distal end. The ablation catheter assembly including a flexible tip connected to the distal end of the elongated catheter shaft, and a source of conductive fluid coupled to the flexible tip to direct flow of the conductive fluid into contact with the electrode, wherein the conductive fluid provides a conductive path from the electrode to tissue to be ablated.

### CROSS-REFERENCE TO RELATED APPLICATION

This application claims priority to Provisional Application No. 62/173,763, filed Jun. 10, 2015, which is herein incorporated by reference in its entirety.

### TECHNICAL FIELD

The present disclosure relates to ablation catheter systems. More specifically, the disclosure relates to ablation catheters and ablation catheter tips.

### BACKGROUND

Supraventricular tachycardia, ventricular tachycardia, and atrial fibrillation are conditions in the heart generally referred to as arrhythmias. During an arrhythmia, abnormal electrical signals, generated in the endocardial tissue, cause irregular beating of the heart. One method of treating these arrhythmias involves creating lesions within the chambers of the heart on the endocardium. These lesions are intended to stop the irregular beating of the heart by creating barriers between regions of the tissue. The barriers halt the passage through the heart of the abnormal currents generated in the endocardium.

Energy, such as radio frequency (RF) energy, can be used to ablate tissue in the heart to form the lesion barriers and stop the flow of abnormal currents. One apparatus for performing ablation is an ablation catheter having an ablative catheter tip.

An electrode at the tip of an ablation catheter is placed inside the body and on the tissue to be ablated. A power supply generates electrical power, such as radio frequency current, that is communicated between the electrode and a return electrode such that the energy ablates the tissue in the vicinity of the electrode.

Problems associated with ablation include clotting of the blood and “popping,” which refers to explosions in the body's tissue during ablation. Clotting has been observed to be correlated with denaturizing the blood with high temperature and/or high current density areas in the vicinity of the catheter tip. The clots or coagulum are undesirable because they may travel through the blood stream and cause one or more embolic events. Popping is undesirable because it causes irregularities, such as tears, in the ablated tissue and it becomes more difficult to control which tissue is ablated. In addition, the force of the popping explosions can cause and disperse clots or coagulum.

### SUMMARY

In an Example 1, an ablation catheter assembly includes a source of ablation energy coupled to a lead wire disposed within an elongated catheter shaft having a proximal end and a distal end. The lead wire has an electrode disposed at the distal end and the ablation catheter assembly includes a flexible tip connected to the distal end of the elongated catheter shaft, and a source of conductive fluid coupled to the flexible tip to direct flow of the conductive fluid into contact with the electrode, wherein the conductive fluid provides a conductive path from the electrode to tissue to be ablated.

In an Example 2, the ablation catheter assembly according to Example 1, wherein the elongated catheter shaft and the flexible tip are separately steerable and comprising a catheter steering element to steer the elongated catheter shaft and a tip steering element to steer the flexible tip.

In an Example 3, the ablation catheter assembly according to any of Examples 1 and 2, wherein the flexible tip is compliant to conform to the shape of the tissue to be ablated.

In an Example 4, the ablation catheter assembly according to any of Examples 1-3, wherein the flexible tip extends distal of the electrode to engage tissue around the tissue to be ablated and to maintain the electrode away from the tissue to be ablated.

In an Example 5, the ablation catheter assembly according to any of Examples 1-4, wherein the electrode is disposed within the flexible tip such that at least a portion of the flexible tip is disposed between the electrode and the tissue to be ablated.

In an Example 6, the ablation catheter assembly according to any of Examples 1-5, wherein the flexible tip includes a porous material to absorb and pass the conductive fluid and provide the conductive path through the flexible tip.

In an Example 7, the ablation catheter assembly according to any of Examples 1-6, wherein the flexible tip has holes extending through the flexible tip, such that the conductive fluid passes through the flexible tip to provide the conductive path through the flexible tip.

In an Example 8, the ablation catheter assembly according to any of Examples 1-7, comprising a cover material on the flexible tip, wherein the cover material passes the conductive fluid to provide the conductive path through the cover material and the flexible tip and the cover material is at least one of internal to the flexible tip and external to the flexible tip.

In an Example 9, a method of ablating tissue in a patient using an ablation catheter assembly including a lead wire disposed within an elongated catheter shaft having a proximal end and a distal end. The lead wire has an electrode disposed at the distal end and the method includes conforming a flexible tip at the distal end of the elongated catheter shaft to the tissue to be ablated, supplying conductive fluid to the flexible tip and into contact with the electrode, and supplying ablation energy to the lead wire and the electrode, wherein the conductive fluid provides a conductive path from the electrode to the tissue to be ablated.

In an Example 10, the method of Example 9, including steering the elongated catheter shaft to the tissue to be ablated, and steering the flexible tip into position at the tissue to be ablated to provide at least one of spot ablation and linear ablation.

In an Example 11, the method of any of Examples 9 and 10, comprising spacing the electrode away from interior wall surfaces of the flexible tip at the distal end of the elongated catheter shaft.

In an Example 12, the method of any of Examples 9-11, comprising absorbing the conductive fluid in porous material that is part of the flexible tip to provide the conductive path through the flexible tip to the tissue to be ablated.

In an Example 13, the method of any of Examples 9-12, comprising providing holes through the flexible tip, and passing the conductive fluid through the flexible tip to provide the conductive path through the flexible tip to the tissue to be ablated.

In an Example 14, the method of any of Examples 9-13, comprising covering the flexible tip with material on at least one of inside the flexible tip and outside the flexible tip, and passing the conductive fluid through the material to provide the conductive path through the flexible tip to the tissue to be ablated.

In an Example 15, the method of any of Examples 9-11, wherein conforming the flexible tip comprises extending the flexible tip out of the distal end of the elongated catheter shaft to engage tissue around the tissue to be ablated and to enclose the electrode between the elongated catheter shaft, the flexible tip, and the tissue to be ablated.

In an Example 16, an ablation catheter assembly comprising an elongated catheter shaft having a proximal end and a distal end, a flexible tip connected to the distal end of the catheter shaft, a lead wire disposed within the catheter shaft, an electrode disposed at the distal end, a source of ablation energy coupled to the lead wire, and a source of conductive fluid coupled to the flexible tip to direct flow of the conductive fluid into contact with the electrode, wherein the conductive fluid provides a conductive path from the electrode to tissue to be ablated.

In an Example 17, the ablation catheter assembly of Example 16, wherein the elongated catheter shaft and the flexible tip are separately steerable and comprising a catheter steering element to steer the elongated catheter shaft and a tip steering element to steer the flexible tip.

In an Example 18, the ablation catheter assembly of Example 16, wherein the flexible tip is compliant to conform to the shape of the tissue to be ablated.

In an Example 19, the ablation catheter assembly of Example 16, wherein the flexible tip extends distal of the electrode to engage tissue surrounding the tissue to be ablated and to maintain the electrode away from the tissue to be ablated.

In an Example 20, the ablation catheter assembly of Example 16, wherein the electrode is disposed within the flexible tip such that at least a portion of the flexible tip is disposed between the electrode and the tissue to be ablated.

In an Example 21, the ablation catheter assembly of Example 16, wherein the flexible tip includes a porous material to absorb and pass the conductive fluid and provide the conductive path through the flexible tip.

In an Example 22, the ablation catheter assembly of Example 16, wherein the flexible tip has holes extending through the flexible tip, such that the conductive fluid passes through the flexible tip to provide the conductive path through the flexible tip.

In an Example 23, the ablation catheter assembly of Example 22, comprising a cover material on the flexible tip, wherein the cover material passes the conductive fluid to provide the conductive path through the cover material and the flexible tip.

In an Example 24, the ablation catheter assembly of Example 23, wherein the cover material is at least one of internal to the flexible tip and external to the flexible tip.

In an Example 25, the ablation catheter assembly of Example 16, comprising mini electrodes situated on the flexible tip to provide tissue contact feedback.

In an Example 26, an ablation catheter assembly includes an elongated catheter shaft having a proximal end and a distal end, a tip connected to the distal end of the catheter shaft and configured to pass conductive fluid, a lead wire disposed within the catheter shaft, an electrode disposed at the distal end within the tip and spaced away from the interior wall surfaces of the tip, a source of ablation energy coupled to the lead wire, a cover material disposed about the tip and configured to pass the conductive fluid, and a source of the conductive fluid coupled to the tip to direct flow of the conductive fluid into contact with the electrode, wherein the conductive fluid provides a conductive path from the electrode and through the tip and the cover material to tissue to be ablated.

In an Example 27, the ablation catheter assembly of Example 26, wherein the tip has holes extending through the tip to pass the conductive fluid and provide the conductive path through the tip.

In an Example 28, the ablation catheter assembly of Example 26, wherein the cover material is at least one of disposed outside the tip and disposed inside the tip.

In an Example 29, a method of ablating tissue in a patient comprising providing a flexible tip at a distal end of an elongated catheter shaft, providing a lead wire disposed within the elongated catheter shaft and an electrode disposed at the distal end, conforming the flexible tip to the tissue to be ablated, supplying conductive fluid to the flexible tip and into contact with the electrode, and supplying ablation energy to the lead wire and the electrode, wherein the conductive fluid provides a conductive path from the electrode to the tissue to be ablated.

In an Example 30, the method of Example 29, comprising steering the elongated catheter shaft to the tissue to be ablated, and steering the flexible tip into position at the tissue to be ablated to provide at least one of spot ablation and linear ablation.

In an Example 31, the method of Example 29, wherein conforming the flexible tip to the tissue to be ablated comprises at least one of extending the flexible tip out of the distal end of the elongated catheter shaft to engage tissue encircling the tissue to be ablated and to enclose the electrode between the flexible tip and the tissue to be ablated, and vacuuming blood away from the electrode and from between the flexible tip and the tissue to be ablated.

In an Example 32, the method of Example 29, wherein the electrode is spaced away from the interior wall surfaces of the flexible tip.

In an Example 33, the method of Example 29, comprising absorbing the conductive fluid in porous material that is part of the flexible tip to provide the conductive path through the flexible tip to the tissue to be ablated.

In an Example 34, the method of Example 29, comprising providing holes through the flexible tip, and passing the conductive fluid through the flexible tip to provide the conductive path through the flexible tip to the tissue to be ablated.

In an Example 35, the method of Example 29, comprising covering the flexible tip with material on at least one of inside the flexible tip and outside the flexible tip, and passing the conductive fluid through the material to provide the conductive path through the flexible tip to the tissue to be ablated.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 is a diagram illustrating an ablation catheter assembly that includes a flexible catheter tip for treating tissue to be treated, according to embodiments described in the disclosure.

FIG. 2 is a diagram illustrating the catheter tip with the lead wire and the electrode inside the catheter tip, according to embodiments described in the disclosure.

FIG. 3 is a diagram illustrating the catheter tip bending along its length L, according to some embodiments described in the disclosure.

FIG. 4 is a diagram illustrating the catheter tip being steered or bent to form an acute angle A at or toward the proximal end of the catheter tip, according to embodiments described in the disclosure.

FIG. 5 is a diagram illustrating a pencil shaped catheter tip, according to embodiments described in the disclosure.

FIG. 6 is a diagram illustrating a spherical or ball shaped catheter tip, according to embodiments described in the disclosure.

FIG. 7 is a diagram illustrating a spatula or paint brush shaped catheter tip, according to embodiments described in the disclosure.

FIG. 8 is a diagram illustrating the catheter tip having slots for passing the conductive fluid, according to embodiments described in the disclosure.

FIG. 9 is a diagram illustrating the catheter tip having slots for passing the conductive fluid, according to embodiments described in the disclosure.

FIG. 10 is a diagram illustrating another ablation catheter assembly including a flexible catheter tip, according to embodiments described in the disclosure.

FIG. 11 is a diagram illustrating the ablation catheter assembly with the catheter tip tucked inside the elongated catheter shaft, according to embodiments described in the disclosure.

FIG. 12 is a diagram illustrating the catheter tip deployed beyond the distal end of the electrode, according to embodiments described in the disclosure.

FIG. 13 is a diagram illustrating the catheter tip steered or bent to engage the tissue, according to embodiments described in the disclosure.

FIG. 14 is a flow chart diagram illustrating a method of treating tissue in a patient using an ablation catheter assembly, according to embodiments described in the disclosure.

FIG. 15 is a flow chart diagram illustrating another method of treating tissue in a patient using an ablation catheter assembly, according to embodiments described in the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

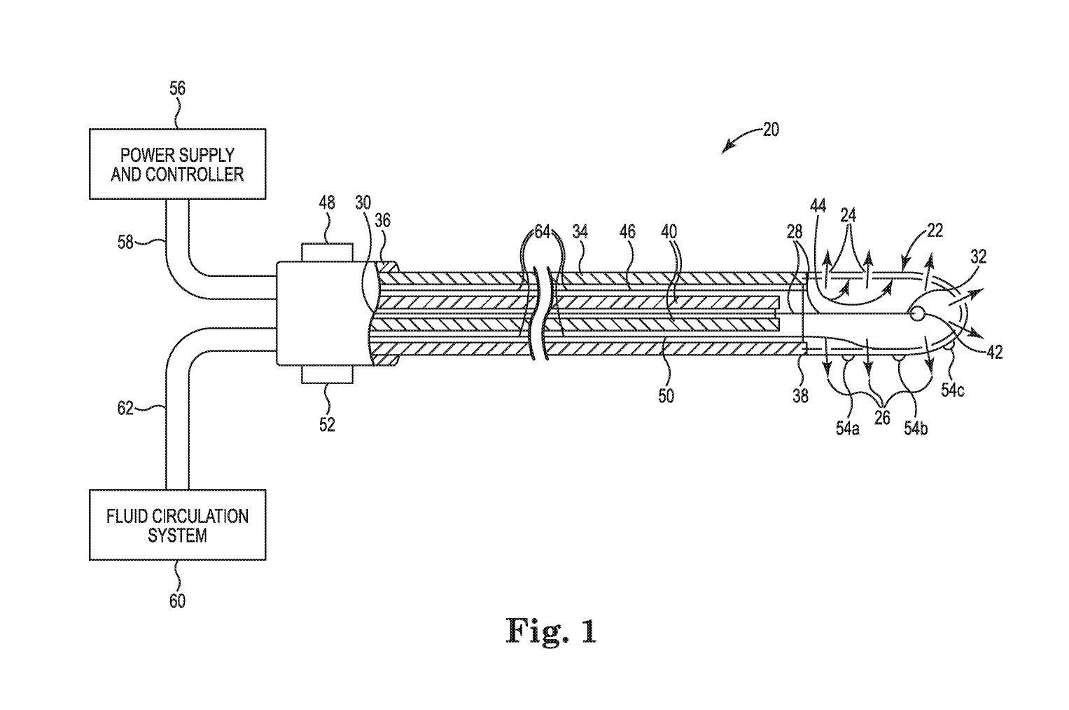

FIG. 1 is a diagram illustrating an ablation catheter assembly 20 that includes a flexible catheter tip 22 for treating tissue to be treated, according to embodiments described in the disclosure. The catheter tip 22 is porous, having holes 24 that extend through the catheter tip 22 to pass conductive fluid 26 to the tissue to be treated. The conductive fluid 26 provides a conductive pathway through the catheter tip 22 and to the tissue. Treatment of the tissue can include ablation of the tissue and/or otherwise creating lesions in the tissue. In some embodiments, the conductive fluid 26 includes a saline solution, which may, in embodiments, have an increased density, such as from 0.1% to 1.0% sodium chloride in water.

The catheter tip 22 is flexible, such that, in at least some embodiments, the catheter tip 22 bends or it can be steered or bent to contact the tissue and to conform the catheter tip 22 to the tissue to be treated. Also, in some embodiments, the catheter tip 22 is flexible, such that the catheter tip 22 is soft and sponge-like for contacting the tissue and conforming the catheter tip 22 to the tissue to be treated.

The ablation catheter assembly 20 includes a lead wire 28 having a proximal end 30 and a distal end 32, which extends through an elongated catheter shaft 34 having a shaft proximal end 36 and a shaft distal end 38. The catheter tip 22 is attached to the shaft distal end 38 and the lead wire 28 extends through a wire lumen 40 in the elongated catheter shaft 34 and into the catheter tip 22. An electrode 42 is attached to the distal end 32 of the lead wire 28 in the catheter tip 22 at the shaft distal end 38. The electrode 42 is spaced away from the inside surfaces 44 of the catheter tip 22. In embodiments, the electrode 42 may include stainless steel, platinum, and/or the like.

In embodiments, the elongated catheter shaft 34 and the catheter tip 22 are each independently steerable or bendable. A shaft steering element 46 may be connected to the shaft distal end 38 and to a shaft steering control 48 located at the shaft proximal end 36. Also, a catheter tip steering element 50 may be connected to the catheter tip 22 and to a catheter tip steering control 52 at the shaft proximal end 36. Using the shaft steering control 48, the elongated catheter shaft 34 can be steered or bent to the tissue to be treated and using the catheter tip steering control 52 the catheter tip 22 can be steered or bent to contact the tissue and conform to the tissue to be treated. The catheter tip 22 can be aligned to perform either spot ablation or linear ablation. In some embodiments, the shaft steering element 46 includes a wire for steering or bending the elongated catheter shaft 34. In some embodiments, the shaft steering element 46 includes a ribbon for steering or bending the elongated catheter shaft 34. In some embodiments, the shaft steering element 46 includes tubing for steering or bending the elongated catheter shaft 34. In some embodiments, the catheter tip steering element 50 includes a wire for steering or bending the catheter tip 22. In some embodiments, the catheter tip steering element 50 includes a ribbon for steering or bending the catheter tip 22. In some embodiments, the catheter tip steering element 50 includes tubing for steering or bending the catheter tip 22.

In some embodiments, the ablation catheter assembly 20 includes mini electrodes 54 situated on at least one of the catheter tip 22 and the elongated catheter shaft 34. The mini-electrodes 54 can be used to indicate contact characteristics of the catheter tip 22 on the surface of the tissue. In some embodiments, three mini-electrodes 54a-54c are provided on the catheter tip 22. In other embodiments, four or more mini-electrodes may be provided on the catheter tip 22.

The ablation catheter assembly 20 includes a power supply and controller 56 that is electrically coupled to the proximal end 30 of the lead wire 28 via conductive pathway 58. The power supply and controller 56 includes a source of energy that provides energy to the lead wire 28 and the electrode 42. The power supply and controller 56 controls the characteristics of the energy delivered to the electrode 42 to provide treatment to the tissue. In some embodiments, the power supply and controller 56 provides high frequency energy, such as RF energy, to the electrode 42 to ablate tissue and/or otherwise create lesions in the tissue.

The ablation catheter assembly 20 also includes a fluid circulation system 60 that is fluidically coupled to the elongated catheter shaft 34 at the shaft proximal end 36 via fluidic coupling 62. The elongated catheter shaft 34 includes fluid conduits 64 that receive the conductive fluid 26 from the fluid circulation system 60 and pass the conductive fluid 26 through the elongated catheter shaft 34 to the catheter tip 22. The catheter tip 22 receives the conductive fluid 26 and absorbs and/or passes the conductive fluid 26, such that the conductive fluid 26 flows through the catheter tip 22 to the tissue to be treated. The conductive fluid 26 contacts the electrode 42 and conducts the electrical energy provided to the electrode 42 by the power supply and controller 56 to treat the tissue to be treated.

In operation, the catheter tip 22 is applied against the tissue to be treated and the fluid circulation system 60 provides the conductive fluid 26 through the elongated catheter shaft 34 to the catheter tip 22. The catheter tip 22 receives the conductive fluid 26 and passes the conductive fluid 26 through the catheter tip 22 to the tissue. The power supply and controller 56 provides electrical energy to the electrode 42 and the conductive fluid 26 conducts the electrical energy and provides a conductive path for the electrical energy to the tissue. The conductive fluid 26 and the catheter tip 22 contact the tissue, but the electrode 42 does not touch the tissue to be treated, which reduces or eliminates clotting and popping. Treatment of the tissue can include spot or linear ablation of the tissue and/or otherwise creating lesions in the tissue.

The ablation catheter assembly 20 can be used to effectively treat arrhythmias, including supraventricular tachycardia, ventricular tachycardia, and atrial fibrillation. By applying the energy to the tissue via the conductive fluid 26 and the flexible catheter tip 22, the ablation catheter assembly 20 may minimize complications in patients undergoing ablation therapy by reducing or eliminating clotting and popping.

FIG. 2 is a diagram illustrating the catheter tip 22 with the lead wire 28 and the electrode 42 inside the catheter tip 22, according to embodiments described in the disclosure. The catheter tip 22 includes the holes 24 through which the conductive fluid 26 passes to contact the tissue to be treated. However, in other embodiments, the catheter tip 22 can be a porous foam or sponge-like material that may not include distinct holes 24, and yet still allows the conductive fluid 26 to be absorbed and/or pass through the catheter tip 22.

The holes 24 in the catheter tip 22 regulate the flow of the conductive fluid 26 through the catheter tip 22. The number, shape, size, and position of the holes 24 in the catheter tip 22 can be adjusted to satisfy treatment needs. In the illustrated embodiments, the holes 24 in catheter tip 22 are circular and evenly spaced apart. However, in other embodiments, the holes 24 can be different in number, shape, size, and/or position to regulate the flow of the conductive fluid 26 through the catheter tip 22. In embodiments, the holes 24 are on only one or two sides of the catheter tip 22. In embodiments, the holes 24 are larger or smaller at one end of the catheter tip 22 than at the opposite end.

The catheter tip 22 is flexible for contacting the surface of the tissue to be treated. In some embodiments, the catheter tip 22 can be steered or bent to contact the tissue. In some embodiments, the catheter tip 22 can be pushed or pressure can be applied to force the catheter tip 22 to bend and conform to the tissue. In some embodiments, the catheter tip 22 can be soft and sponge-like for contacting the tissue and conforming to the tissue to be treated.

The catheter tip 22 can include at least one of a non-metallic material, such as plastic or ceramic, and a metallic material. Also, the catheter tip 22 can include material that absorbs and/or passes the conductive fluid 26. In some embodiments, the catheter tip 22 includes a porous material that absorbs and/or passes the conductive fluid 26. In some embodiments, the catheter tip 22 includes a bio-absorbable material that absorbs and/or passes the conductive fluid 26. In some embodiments, the catheter tip 22 includes plant fiber, such as cotton and paper. In some embodiments, the catheter tip 22 includes at least one of a foam and sponge-like material that absorbs and/or passes the conductive fluid.

Optionally, the catheter tip 22 can include a cover 70 (indicated in dashed lines) that covers the holes 24 of the catheter tip 22. The cover 70 can be installed on the outside, over the catheter tip 22, or on the inside of the catheter tip 22. The cover 70 covers the holes 24 to regulate the flow of the conductive fluid 26. The cover 70 is porous to allow the conductive fluid 26 to be absorbed and/or pass through the cover 70. In some embodiments, the cover 70 includes a bio-absorbable material. In some embodiments, the cover 70 includes plant fiber, such as cotton and paper. In some embodiments, the cover 70 includes at least one of a foam and sponge-like material. In some embodiments, the cover 70 is a fabric cover, such as a cloth cover.

According to embodiments, the catheter tip 22 has a length L and a diameter D. The length L extends from the proximal end 72 of the catheter tip 22 to the distal end 74 of the catheter tip 22. In some embodiments, the length L is from 4 to 5 millimeters (mm) and, in some embodiments, the diameter D is from 2 to 3 mm.

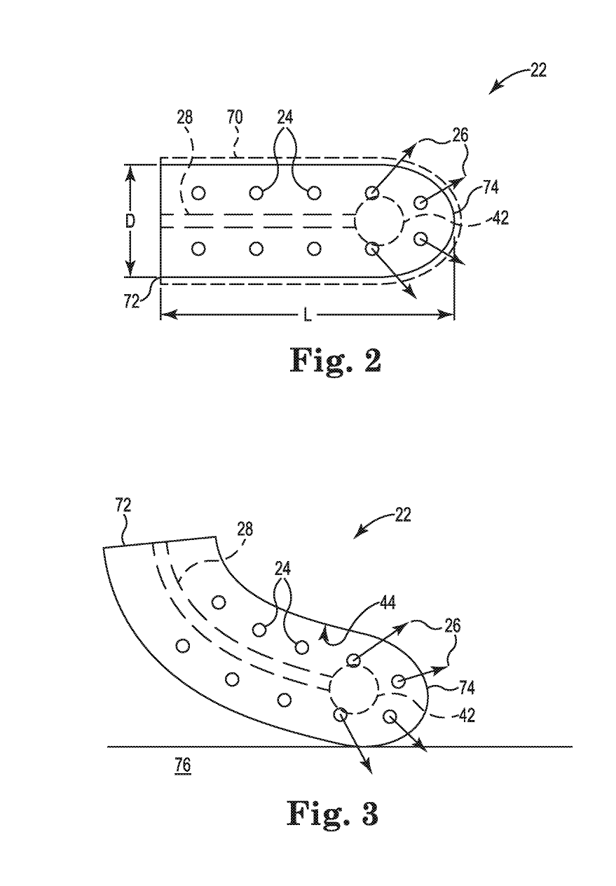

FIG. 3 is a diagram illustrating the catheter tip 22 bending along its length L, according to some embodiments described in the disclosure. The catheter tip 22 bends to at least partially conform the catheter tip 22 to the tissue 76 to be treated. The lead wire 28 bends with the catheter tip 22 and the electrode 42 remains at or toward the distal end 74 of the catheter tip 22. In some embodiments, the electrode 42 is forced against the inside surface 44 of the catheter tip 22 as the catheter tip 22 bends, which bends the lead wire 28.

In some embodiments, the catheter tip 22 is independently steered to bend the catheter tip 22. In some embodiments, the catheter tip 22 can be pushed against the tissue 76 or pressure can be applied to force the catheter tip 22 against the tissue 76, which bends the catheter tip 22.

Bending the catheter tip 22 can provide better alignment of the catheter tip 22 with the tissue 76 to be treated and a more controlled treatment.

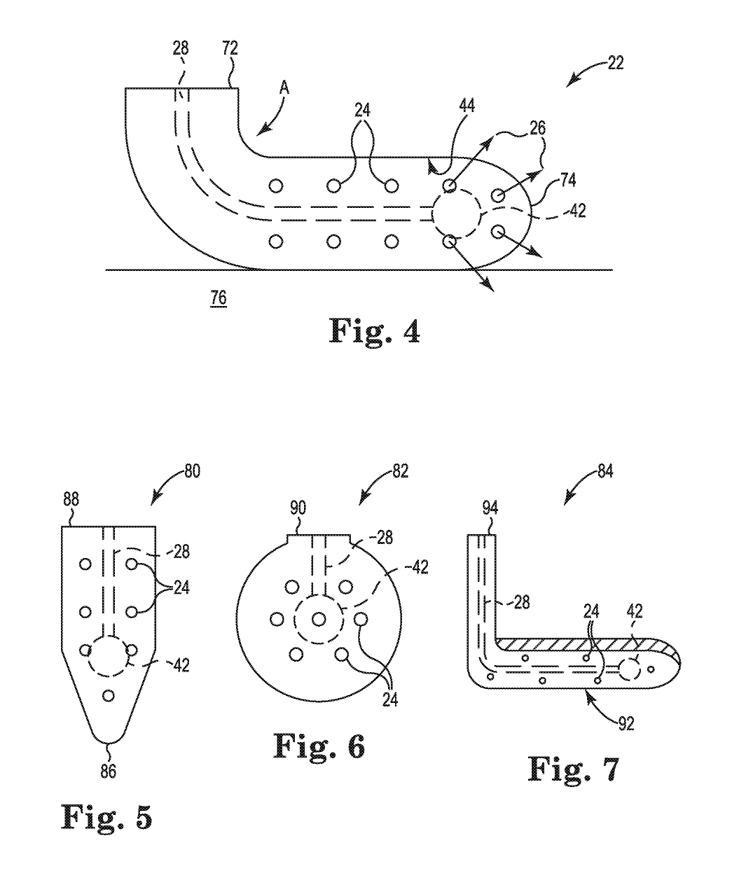

FIG. 4 is a diagram illustrating the catheter tip 22 being steered or bent to form an acute angle A at or toward the proximal end 72 of the catheter tip 22, according to embodiments described in the disclosure. The catheter tip 22 can be steered or bent to form the more acute angle A for linear ablation procedures along the tissue 76.

As described above, the lead wire 28 bends with the catheter tip 22 and the electrode 42 remains at or toward the distal end 74 of the catheter tip 22. In some embodiments, the electrode 42 is forced against the inside surface 44 of the catheter tip 22 as the catheter tip 22 bends, which bends the lead wire 28. In some embodiments, the catheter tip 22 is independently steered to bend the catheter tip 22. In some embodiments, the catheter tip 22 can be pushed against the tissue 76 or pressure can be applied to force the catheter tip 22 against the tissue 76, which bends the catheter tip

FIGS. 5-7 are diagrams illustrating catheter tips 80, 82, and 84 that are similar to catheter tip 22 with the exception of having different shapes than the catheter tip 22. In other embodiments, the catheter tips can have other shapes.

FIG. 5 is a diagram illustrating a pencil-shaped catheter tip 80, according to embodiments described in the disclosure. The catheter tip 80 is flexible and includes the holes 24 and the lead wire 28 connected to the electrode 42. The catheter tip 80 is similar to the catheter tip 22, with the exception of the shape of the catheter tips 22 and 80.

The pencil-shaped catheter tip 80 is sharpened or tapers to a rounded point at the distal end 86, and it attaches to the elongated catheter shaft 34 at the proximal end 88. The rounded distal end 86 can be used advantageously for providing spot ablation.

FIG. 6 is a diagram illustrating a spherical or ball-shaped catheter tip 82, according to embodiments described in the disclosure. The catheter tip 82 is flexible and includes the holes 24 and the lead wire 28 connected to the electrode 42. The catheter tip 82 is similar to the catheter tip 22, with the exception of the shape of the catheters.

The ball-shaped catheter tip 80 is spherical in shape and attaches to the elongated catheter shaft 34 at one end 90. The ball-shaped catheter tip 82 can be used for providing circular or arc shaped ablation on the tissue to be treated.

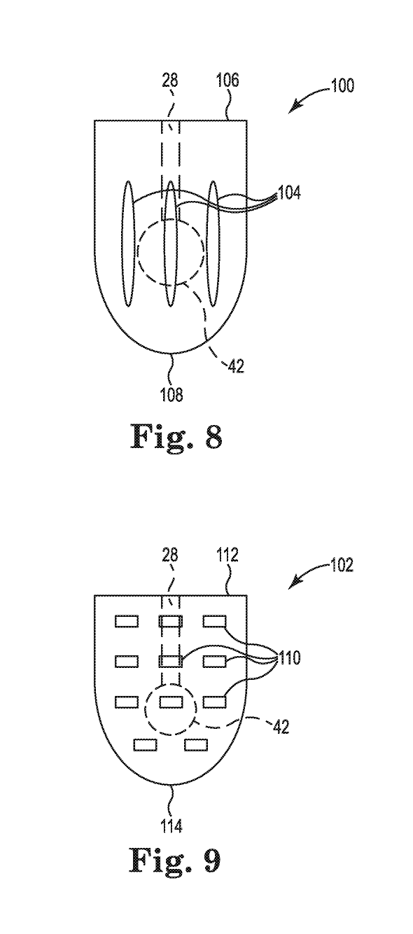

FIG. 7 is a diagram illustrating a spatula- or paint-brush shaped catheter tip 84, according to embodiments described in the disclosure. The catheter tip 84 is flexible and includes the holes 24 and the lead wire 28 connected to the electrode 42. The catheter tip 84 is similar to the catheter tip 22, with the exception of the shape of the catheter tips 22 and 84.

The spatula- or paint-brush shaped catheter tip 84 is flat along the bottom 92 and attaches to the elongated catheter shaft 34 at the proximal end 94. The spatula- or paint-brush shaped catheter tip 84 can be used for providing a rectangular or square shaped ablation on the tissue to be treated.

FIGS. 8 and 9 are diagrams illustrating catheter tips 100 and 102 that are similar to the catheter tip 22 with the exception of having different holes than the holes 24 of the catheter tip 22. The flow of the conductive fluid 26 through the catheter tips 100 and 102 can be regulated by the number, size, shape, and/or locations of the holes 24 in the catheter tips. In other embodiments, the holes can have other shapes.

FIG. 8 is a diagram illustrating the catheter tip 100 having slots 104 for passing the conductive fluid 26, according to embodiments described in the disclosure. The slots 104 are cut into, and extend through, the catheter tip 100 to pass the conductive fluid 26. The catheter tip 100 is flexible and includes the lead wire 28 connected to the electrode 42. The catheter tip 100 is similar to the catheter tip 22, with the exception of the slots 104 being different than the holes 24.

The catheter tip 100 has a proximal end 106 and a distal end 108, where the slots 104 extend between the proximal end 106 and the distal end 108. The catheter tip 100 is attached to the elongated catheter shaft 34 at the proximal end 106. In some embodiments, different slots 104 have different sizes around the perimeter of the catheter tip 100. In some embodiments, the catheter tip 100 includes multiple slots 104 between the proximal end 106 and the distal end 108. In some embodiments, the slots 104 have different sizes with larger or smaller slots situated closer to the distal end 108.

FIG. 9 is a diagram illustrating the catheter tip 102 having slots 110 for passing the conductive fluid 26, according to embodiments described in the disclosure. The slots 110 are cut into and extend through the catheter tip 102 to pass the conductive fluid 26. The catheter tip 102 is flexible and includes the lead wire 28 connected to the electrode 42. The catheter tip 102 is similar to the catheter tip 22, with the exception of the slots 110 being different than the holes 24.

The catheter tip 102 has a proximal end 112 and a distal end 114, where the slots 110 are rectangular shaped slots 110 aligned circumferentially and between the proximal end 112 and the distal end 114. The catheter tip 102 is attached to the elongated catheter shaft 34 at the proximal end 112. In some embodiments, different slots 110 have different sizes around the circumference or perimeter of the catheter tip 102. In some embodiments, the slots 110 have different sizes with larger or smaller slots situated closer to the distal end 114.

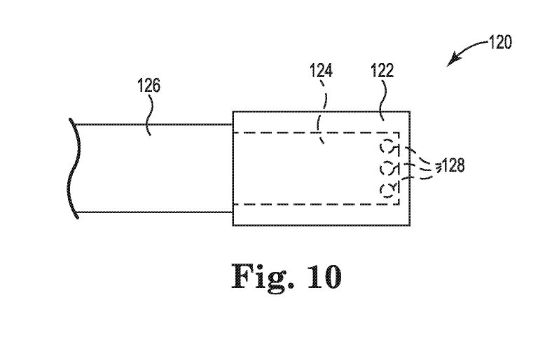

FIG. 10 is a diagram illustrating another ablation catheter assembly 120 including a flexible catheter tip 122, according to embodiments described in the disclosure. The catheter tip 122 is soft and sponge-like for contacting the tissue to be treated and to conform, at least slightly, to the tissue. The catheter tip 122 is porous to absorb and pass the conductive fluid 26 to the tissue to be treated, where the conductive fluid 26 provides the conductive pathway through the catheter tip 122 and to the tissue. The treatment of the tissue can include ablation of the tissue and/or otherwise creating lesions in the tissue.

The catheter assembly 120 includes an ablation electrode 124 connected to an elongated catheter shaft 126. The ablation electrode 124 includes holes 128 through which the conductive fluid 26 flows and the ablation electrode 124 is electrically coupled to the power supply and controller 56. The elongated catheter shaft 126 is fluidically coupled to the fluid circulation system 60 and includes fluid conduits that receive and pass the conductive fluid 26 to the ablation electrode 124 and the holes 128. The elongated catheter shaft 126 is similar to the elongated catheter shaft 34. In some embodiments, the ablation electrode 124 includes stainless steel. In some embodiments, the ablation electrode 124 includes platinum.

The catheter tip 122 is secured to the elongated catheter shaft 126 and/or to the ablation electrode 124. In some embodiments, the catheter tip 122 is secured via one or more of hot plastics, adhesives, and screwing the catheter tip 122 onto the ablation electrode 124.

The catheter tip 122 includes material that absorb and pass the conductive fluid 26. In some embodiments, the catheter tip 122 includes a porous material that absorbs and passes the conductive fluid 26. In some embodiments, the catheter tip 122 includes a bio-absorbable material that absorbs and passes the conductive fluid 26. In some embodiments, the catheter tip 122 includes plant fiber, such as cotton and paper. In some embodiments, the catheter tip 122 includes at least one of a foam material and sponge-like material that absorb and pass the conductive fluid 26.

In some embodiments, the catheter assembly 120 includes mini electrodes (not shown) that are situated on at least one of the catheter tip 122 and the elongated catheter shaft 126. The mini-electrodes can be used to indicate contact characteristics, such as touching and pressure, of the catheter tip 122 on the surface of the tissue. In some embodiments, three mini-electrodes are provided on the catheter tip 122, while in other embodiments, four or more mini-electrodes may be provided.

In operation, the catheter tip 122 is pressed against the tissue to be treated and the fluid circulation system 60 provides the conductive fluid 26 through the elongated catheter shaft 126 to the holes 128 and the catheter tip 122. The catheter tip 122 receives the conductive fluid 26 and absorbs and passes at least some of the conductive fluid 26. The saturated catheter tip 122 touches the tissue, and the power supply and controller 56 provides electrical energy to the ablation electrode 124. The conductive fluid 26 conducts the electrical energy and provides a conductive path for the electrical energy to the tissue. The conductive fluid 26 and the catheter tip 122 contact the tissue, but the ablation electrode 124 does not touch the tissue, which may reduce or eliminate clotting and/or popping. Treatment of the tissue can include spot or linear ablation of the tissue and/or otherwise creating lesions in the tissue.

The ablation catheter assembly 120 can be used to effectively treat arrhythmias, including supraventricular tachycardia, ventricular tachycardia, and atrial fibrillation. By applying the energy to the tissue with the conductive fluid 26 and the flexible catheter tip 122, the ablation catheter assembly 120 may minimize complications in patients undergoing ablation therapy by reducing or eliminating clotting and/or popping.

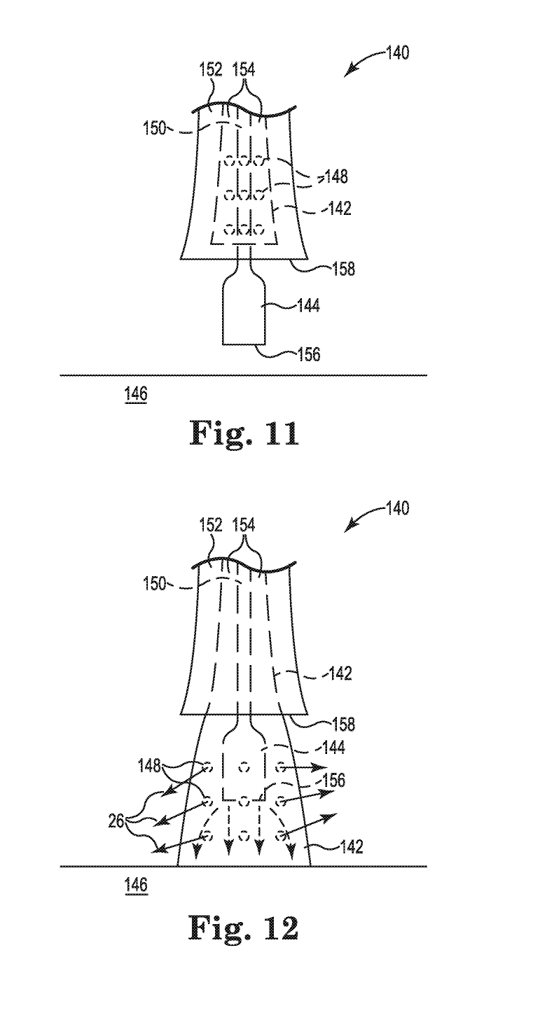

FIGS. 11-13 are diagrams illustrating another ablation catheter assembly 140 including a flexible catheter tip 142, according to embodiments described in the disclosure. In this ablation catheter assembly 140, the catheter tip 142 is not situated between the electrode 144 and the tissue 146 to be treated. Instead, the catheter tip 142 receives and guides the conductive fluid 26 to the tissue 146 to be treated. The catheter tip 142 maintains the electrode 144 away from the tissue 146 and the conductive fluid 26 provides the conductive pathway to the tissue 146 from the electrode 144. Treatment of the tissue can include ablation of the tissue and/or otherwise creating lesions in the tissue. In some embodiments, as illustrated in FIGS. 11-13, the catheter tip 142 is porous having holes 148 that extend through the catheter tip 142 to pass the conductive fluid 26.

The catheter tip 142 is flexible, such that, in at least some embodiments, the catheter tip 142 bends or it can be steered or bent to contact the tissue 146 and to conform the catheter tip 142 to the tissue 146 to be treated. Also, in some embodiments, the catheter tip 142 is flexible, such that the catheter tip 142 is soft and sponge-like for contacting the tissue 146 and conforming the catheter tip 142 to the tissue 146 to be treated.

The catheter assembly 140 includes the electrode 144 connected to a lead wire 150 that extends through an elongated catheter shaft 152. The lead wire 150 and the electrode 144 are electrically coupled to the power supply and controller 56. The elongated catheter shaft 152 is fluidically coupled to the fluid circulation system 60 and includes fluid conduits 154 that receive and pass the conductive fluid 26 to the electrode 144 and the tissue 146. In embodiments, the electrode 144 includes stainless steel, platinum, and/or the like.

In embodiments, the catheter tip 142 is slidably engaged in the elongated catheter shaft 152, such that the catheter tip 142 can be slid out of the elongated catheter shaft 152 and past the distal end 156 of the electrode 144 to form a skirt around the electrode 144 and to engage the tissue 146. With the catheter tip 142 extended past the distal end 156 of the electrode 144, the catheter tip 142 maintains the electrode 144 away from the tissue 146 and the conductive fluid 26 provides the conductive path from the electrode 144 to the tissue 146.

In other embodiments, the elongated catheter shaft 152 is similar to the elongated catheter shaft 34 and the catheter tip 142 is attached to the distal end 158 of the elongated catheter shaft 152. In embodiments, the catheter tip 142 can be secured to the elongated catheter shaft 152. In some embodiments, the catheter tip 142 is secured via one or more of hot plastics, adhesives, and screwing the catheter tip 142 onto the elongated catheter shaft 152.

The catheter tip 142 can include porous material that absorbs and passes the conductive fluid 26. In some embodiments, the catheter tip 142 includes a bio-absorbable material. In some embodiments, the catheter tip 142 includes plant fiber, such as cotton and paper. In some embodiments, the catheter tip 142 includes at least one of a foam material and sponge-like material. In some embodiments, the catheter tip 142 includes one or more of plastic and rubber.

In some embodiments, the elongated catheter shaft 152 and the catheter tip 142 are each independently steerable or bendable. The shaft steering element 46 can be connected to the shaft distal end 158 and to the shaft steering control 48. Also, the catheter tip steering element 50 can be connected to the catheter tip 142 and to the catheter tip steering control 52. Using the shaft steering control 48, the elongated catheter shaft 152 can be steered or bent to the tissue 146 and using the catheter tip steering control 52 the catheter tip 142 can be steered or bent to contact the tissue 146 and conform to the tissue 146.

In some embodiments, the catheter assembly 140 includes mini electrodes (not shown) that are situated on at least one of the catheter tip 142 and the elongated catheter shaft 152. The mini-electrodes can be used to indicate contact characteristics, such as touching and pressure, of the catheter tip 142 on the tissue 146. In some embodiments, three mini-electrodes are provided on the catheter tip 142, while, in other embodiments, four or more mini-electrodes may be provided.

FIG. 11 is a diagram illustrating the ablation catheter assembly 140 with the catheter tip 142 tucked inside the elongated catheter shaft 152, according to embodiments described in the disclosure. The catheter tip 142 is proximal the distal end 158 of the elongated catheter shaft 152.

FIG. 12 is a diagram illustrating the catheter tip 142 deployed beyond the distal end 156 of the electrode 144, according to embodiments described in the disclosure. The catheter tip 142 extends out of the elongated catheter shaft 152 and past the distal end 156 of the electrode 144 to form the skirt around the electrode 144 and to engage the tissue 146. With the catheter tip 142 extended past the distal end 156 of the electrode 144, the catheter tip 142 maintains the electrode 144 away from the tissue 146 and the conductive fluid 26 provides the conductive path from the electrode 144 to the tissue 146.

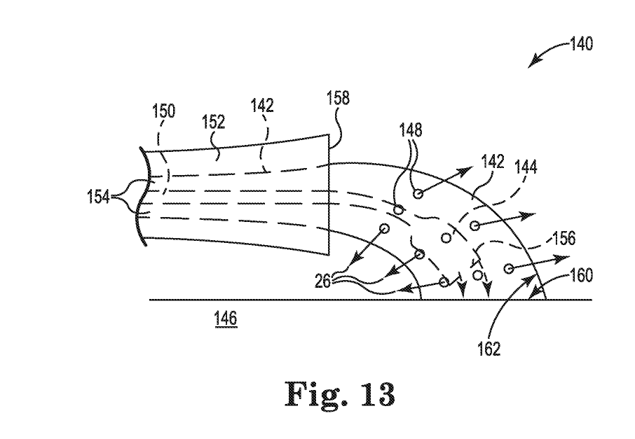

FIG. 13 is a diagram illustrating the catheter tip 142 steered or bent to engage the tissue 146, according to embodiments described in the disclosure. The catheter tip 142 bends to at least partially conform the catheter tip 142 to the tissue 146 to be treated. The lead wire 150 bends with the catheter tip 142 and the electrode 144 remains at or toward the distal end 160 of the catheter tip 142. In some embodiments, the electrode 144 is forced against the inside surface 162 of the catheter tip 142 as the catheter tip 142 bends, which bends the lead wire 150. Bending the catheter tip 142 can provide better alignment of the catheter tip 142 with the tissue 146 to be treated and a more controlled treatment.

In operation, the catheter assembly 140 is aligned next to the tissue 146 to be treated, where the electrode 144 can be spaced away from the tissue 146. The catheter tip 142 is slid out of the elongated catheter shaft 152 to deploy the distal end 160 of the catheter tip 142 beyond the distal end 156 of the electrode 144. The catheter tip 142 extends out of the elongated catheter shaft 152 and past the distal end 156 of the electrode 144 to form the skirt around the electrode 144 and to engage the tissue 146, as illustrated in FIGS. 12 and 13. In some embodiments, after the catheter tip 142 forms the skirt around the electrode 144 and engages the tissue 146, the blood and/or other material are vacuumed out from inside the skirt.

The fluid circulation system 60 provides the conductive fluid 26 through the elongated catheter shaft 154 to the skirt and the tissue 146. The catheter tip 142 receives the conductive fluid 26 and guides the conductive fluid 26 to the tissue 146. This provides the skirt with the conductive fluid 26 and at least dilutes the remaining blood and other material inside the skirt.

The power supply and controller 56 provides electrical energy to the electrode 144 and the conductive fluid 26 conducts the electrical energy to provide a conductive path for the electrical energy from the electrode 144 to the tissue 146. The conductive fluid 26 contacts the tissue, but the electrode 144 does not touch the tissue 146, which reduces or eliminates clotting and popping.

The ablation catheter assembly 140 can be used to effectively treat arrhythmias, including supraventricular tachycardia, ventricular tachycardia, and atrial fibrillation. By applying the energy to the tissue 146 via the conductive fluid 26, the ablation catheter assembly 140 minimizes complications in patients undergoing ablation therapy by reducing or eliminating clotting and popping.

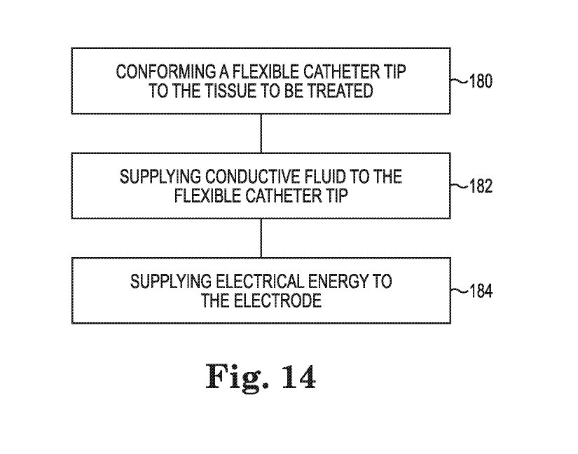

FIG. 14 is a flow chart diagram illustrating a method of treating tissue in a patient using an ablation catheter assembly, such as one of the ablation catheter assemblies 20, 120, and 140.

At 180, the method includes conforming a flexible catheter tip, such as any of the catheter tips described in this disclosure, to the tissue to be treated. In some embodiments, conforming the flexible catheter tip to the tissue includes pressing the catheter tip against the tissue to bend the catheter tip or spreading-out the soft and sponge-like material of the catheter tip on the tissue. In some embodiments, conforming the flexible catheter tip to the tissue includes steering and bending the elongated catheter shaft and/or the catheter tip into contact with the tissue.

At 182, the method includes supplying the conductive fluid to the flexible tip and into contact with the electrode. The fluid circulation system, such as fluid circulation system 60, provides the conductive fluid through the elongated catheter shaft to the catheter tip. The conductive fluid contacts the electrode, such as one of the electrodes 42, 124, and 144 and the catheter tip receives the conductive fluid and passes or guides at least some of the conductive fluid to the tissue to be treated.

At 184, the method includes supplying electrical energy to the lead wire and the electrode, wherein the conductive fluid provides a conductive path from the electrode to the tissue to be treated.

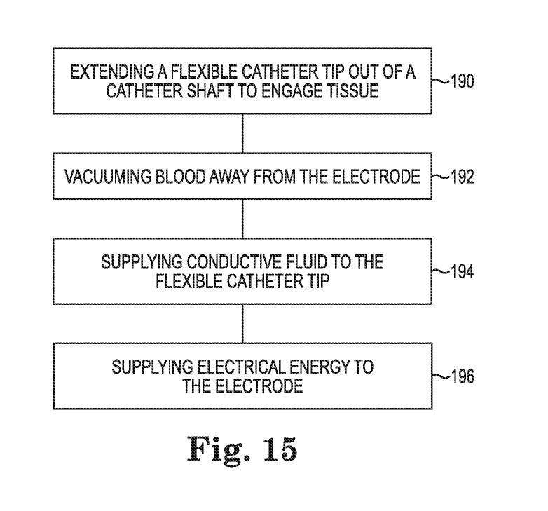

FIG. 15 is a flow chart diagram illustrating another method of treating tissue in a patient using an ablation catheter assembly, such as the ablation catheter assembly 140.

At 190, the method includes extending the flexible catheter tip 142 out of the distal end 158 of the elongated catheter shaft 152 to engage tissue 146 encircling the tissue 146 to be treated, such as by ablation. The flexible catheter tip 142 encloses the electrode 144 between the flexible catheter tip 142 and the tissue 146. The catheter tip 142 is slid out of the elongated catheter shaft 152 to deploy the distal end 160 of the catheter tip 142 beyond the distal end 156 of the electrode 144. The catheter tip 142 extends out of the elongated catheter shaft 152 and past the distal end 156 of the electrode 144 to form a skirt around the electrode 144 and to engage the tissue 146, as illustrated in FIGS. 12 and 13.

At 192, the method includes vacuuming blood away from the electrode 144 and from between the catheter tip 142 and the tissue 146. In other embodiments, the blood is not vacuumed away and this step is not used.

At 194, the method includes supplying the conductive fluid 26 to the catheter tip 142 to contact the electrode 144 and the tissue 146. The fluid circulation system 60 provides the conductive fluid 26 through the elongated catheter shaft 154 to the skirt and the tissue 146. The catheter tip 142 receives the conductive fluid 26 and guides the conductive fluid 26 to the tissue 146, which at least dilutes the remaining blood with the conductive fluid 26.

At 196, the method includes supplying ablation energy to the lead wire 150 and the electrode 144, where the conductive fluid 26 provides a conductive path from the electrode 144 to the tissue 146 to be treated. The power supply and controller 56 provides electrical energy to the electrode 144 and the conductive fluid 26 conducts the electrical energy to provide a conductive path for the electrical energy from the electrode 144 to the tissue 146. The conductive fluid 26 contacts the tissue, but the electrode 144 does not touch the tissue 146, which reduces or eliminates clotting and popping. In some embodiments, the method includes steering the elongated catheter shaft 152 to the tissue 146. In some embodiments, the method includes steering the catheter tip 142 into position at the tissue 146.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An ablation catheter assembly comprising:
an elongated catheter shaft having a proximal end and a distal end;
a flexible tip connected to the distal end of the catheter shaft;
a lead wire disposed within the catheter shaft;
an electrode disposed at the distal end;
a source of ablation energy coupled to the lead wire; and
a source of conductive fluid coupled to the flexible tip to direct flow of the conductive fluid into contact with the electrode, wherein the conductive fluid provides a conductive path from the electrode to tissue to be ablated.

2. The ablation catheter assembly of claim 1, wherein the elongated catheter shaft and the flexible tip are separately steerable and comprising a catheter steering element to steer the elongated catheter shaft and a tip steering element to steer the flexible tip.

3. The ablation catheter assembly of claim 1, wherein the flexible tip is compliant to conform to the shape of the tissue to be ablated.

4. The ablation catheter assembly of claim 1, wherein the flexible tip extends distal of the electrode to engage tissue surrounding the tissue to be ablated and to maintain the electrode away from the tissue to be ablated.

5. The ablation catheter assembly of claim 1, wherein the electrode is disposed within the flexible tip such that at least a portion of the flexible tip is disposed between the electrode and the tissue to be ablated.

6. The ablation catheter assembly of claim 1, wherein the flexible tip includes a porous material to absorb and pass the conductive fluid and provide the conductive path through the flexible tip.

7. The ablation catheter assembly of claim 1, wherein the flexible tip has holes extending through the flexible tip, such that the conductive fluid passes through the flexible tip to provide the conductive path through the flexible tip.

8. The ablation catheter assembly of claim 7, comprising a cover material on the flexible tip, wherein the cover material passes the conductive fluid to provide the conductive path through the cover material and the flexible tip.

9. The ablation catheter assembly of claim 8, wherein the cover material is at least one of internal to the flexible tip and external to the flexible tip.

10. The ablation catheter assembly of claim 1, comprising mini electrodes situated on the flexible tip to provide tissue contact feedback.

11. An ablation catheter assembly comprising:
an elongated catheter shaft having a proximal end and a distal end;
a tip connected to the distal end of the catheter shaft and configured to pass conductive fluid;
a lead wire disposed within the catheter shaft;
an electrode disposed at the distal end within the tip and spaced away from the interior wall surfaces of the tip;
a source of ablation energy coupled to the lead wire;
a cover material disposed about the tip and configured to pass the conductive fluid; and
a source of the conductive fluid coupled to the tip to direct flow of the conductive fluid into contact with the electrode, wherein the conductive fluid provides a conductive path from the electrode and through the tip and the cover material to tissue to be ablated.

12. The ablation catheter assembly of claim 11, wherein the tip has holes extending through the tip to pass the conductive fluid and provide the conductive path through the tip.

13. The ablation catheter assembly of claim 11, wherein the cover material is at least one of disposed outside the tip and disposed inside the tip.

14. A method of ablating tissue in a patient comprising:
providing a flexible tip at a distal end of an elongated catheter shaft;
providing a lead wire disposed within the elongated catheter shaft and an electrode disposed at the distal end;
conforming the flexible tip to the tissue to be ablated;
supplying conductive fluid to the flexible tip and into contact with the electrode; and
supplying ablation energy to the lead wire and the electrode, wherein the conductive fluid provides a conductive path from the electrode to the tissue to be ablated.

15. The method of claim 14, comprising:
steering the elongated catheter shaft to the tissue to be ablated; and
steering the flexible tip into position at the tissue to be ablated to provide at least one of spot ablation and linear ablation.

16. The method of claim 14, wherein conforming the flexible tip to the tissue to be ablated comprises at least one of:
extending the flexible tip out of the distal end of the elongated catheter shaft to engage tissue encircling the tissue to be ablated and to enclose the electrode between the flexible tip and the tissue to be ablated; and
vacuuming blood away from the electrode and from between the flexible tip and the tissue to be ablated.

17. The method of claim 14, wherein the electrode is spaced away from the interior wall surfaces of the flexible tip.

18. The method of claim 14, comprising:
absorbing the conductive fluid in porous material that is part of the flexible tip to provide the conductive path through the flexible tip to the tissue to be ablated.

19. The method of claim 14, comprising:
providing holes through the flexible tip; and
passing the conductive fluid through the flexible tip to provide the conductive path through the flexible tip to the tissue to be ablated.

20. The method of claim 14, comprising:
covering the flexible tip with material on at least one of inside the flexible tip and outside the flexible tip; and
passing the conductive fluid through the material to provide the conductive path through the flexible tip to the tissue to be ablated.
